(19) United States
(12) Patent Application Publication
(10) Pub. No.: US 2015/0074887 A1
(43) Pub. Date: Mar. 19, 2015

(54) Floor Sanitizer for Toilet Area

(71) Applicants: Mark Allan Theuerl, Glendale, AZ (US); Nancy Beth Pluta, Glendale, AZ (US)

(72) Inventors: Mark Allan Theuerl, Glendale, AZ (US); Nancy Beth Pluta, Glendale, AZ (US)

(21) Appl. No.: 14/548,101

(22) Filed: Nov. 19, 2014

Related U.S. Application Data
(60) Provisional application No. 61/906,213, filed on Nov. 19, 2013.

(51) Int. Cl. A61L 2/10; A61L 2/24

(52) U.S. Cl. CPC A61L 2/10; A61L 2/24
USPC 4/222

(57) ABSTRACT

A sanitary device sanitizes the floor area in front of and around a toilet, urinal, or other restroom fixture. An ultraviolet germicidal irradiation (UVGI) lamp or LED irradiates and sanitizes the floor area surrounding the toilet, urinal, or similar fixture. The device operates automatically in response to the detection of motion by a motion sensor.

### CROSS-REFERENCE TO RELATED APPLICATIONS

This application claims the benefit of U.S. Provisional Application No. 61/906,213, filed Nov. 19, 2013, which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to washroom fixtures and in particular to sanitation devices for washroom floors. All too often, when a person uses a toilet, urinal, or other fixture such as a bidet in a public or private restroom, residual drops of urine are left on the floor around the fixture. Drops of urine continue to accumulate with each user and can be tracked on users' shoes, with the accompanying odor and disease causing bacteria, throughout a residence or commercial building. Contrary to popular belief, healthy individuals' urine contains a nonzero level of bacteria, which can reproduce to spread infections and odor when urine remains on a surface; additionally it is not possible to guarantee that all users of a public facility will be free of urinary tract infection, possibly leading to high levels of bacteria in user's urine. Disinfecting the floor area quickly, reliably, and in a completely sanitary manner before the bacteria can be spread to other floor areas can therefore be a challenge.

Despite the usually expected mopping schedule of a commercial building, it is more likely than not that manually sanitizing the restroom floor is fairly infrequent and not nearly in pace with restroom use. Also, most establishments use the same mop for mopping all floor surfaces in the building. Furthermore, when other users are seated on a toilet, they are often forced to mop up urine themselves before lowering their garments, to protect their garments from becoming visibly soiled. This wastes toilet paper and the users often leave the soiled toilet paper on other areas of the floor, adding to the bacteria and odor growth.

A specially designed sanitary device, intended for sanitizing the floor area in front of and around a toilet, urinal, or other restroom fixture, would resolve this problem. The device features a motion sensor, which causes the device to spray a mist of quick-drying sanitizing solution onto the floor, in the area immediately in front of the fixture. The device provides an ultraviolet germicidal irradiation (UVGI) lamp or a spray of sanitizing solution in a refillable or replaceable bladder, bag, or similar reservoir of sanitizing solution.

### SUMMARY OF THE INVENTION

Accordingly, the invention is directed to a sanitary device, which is intended for sanitizing the floor area in front of and around a toilet, urinal, or other restroom fixture. An ultraviolet germicidal irradiation (UVGI) lamp or LED irradiates and sanitizes the floor area surrounding the toilet, urinal, or similar fixture. The device operates automatically in response to the detection of motion by a motion sensor.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitute a part of the specification. They illustrate one embodiment of the invention and, together with the description, serve to explain the principles of the invention.

FIG. 1 is a front view of the first exemplary embodiment in the operational mode, displaying the device 10, the body 10A, the low battery indicator light 10B, the motion sensor 11, the emitter 12, the rotation mechanism 12A, the ball joint 12B, and the top control buttons 13.

FIG. 2 is a side view of the first exemplary embodiment in the operational mode, displaying the device 10, the body 10A, the low battery indicator light 10B, the motion sensor 11, the emitter 12, the rotation mechanism 12A, the ball joint 12B, the top control buttons 13, and the fastener 14.

FIG. 3 is a rear view of the first exemplary embodiment in the operational mode, displaying the device 10, the body 10A, the emitter 12, the rotation mechanism 12A, the ball joint 12B, the top control buttons 13, the fastenerss 14, and the secondary fasteners 14A.

FIG. 4 is a side perspective view of the first exemplary embodiment in the inspection mode, displaying the device 10, the body 10A, the motion sensor 11, the emitter 12, the rotation mechanism 12A, the ball joint 12B, the top control buttons 13, the fasteners 14, and the reservoir 15.

FIG. 5 is a top view of the first exemplary embodiment in the operational mode, displaying the device 10, the body 10A, the motion sensor 11, the top control buttons 13, the fasteners 14, the secondary fasteners 14A, the reservoir 15, the circuit boards 16, and the side control buttons 17.

FIG. 6 is a top view of the first exemplary embodiment in the inspection mode, displaying the device 10, the body 10A, the top control buttons 13, the fasteners 14, the secondary fasteners 14A, the reservoir 15, the printed circuit boards 16, and the side control buttons 17.

FIG. 7 is a lowered perspective view of the second exemplary embodiment configured for a standard toilet, displaying the toilet 700, the toilet bowl 701, the toilet base 702, the device 710, the emitters 711, and the sensors 712.

FIG. 8 is a bottom perspective view of the second exemplary embodiment configured for a standard toilet displaying the toilet 700, the toilet bowl 701, the toilet base 702, the device 710, the emitters 711, and the motion sensors 712.

FIG. 9 is a bottom perspective view of the second exemplary embodiment configured for installation on generic surfaces, displaying the device 900, the fastener surface 910, the battery 911, and the charging terminals 912.

FIG. 10 is an elevated view of the second exemplary embodiment configured for installation on generic surfaces, displaying the device 900, the emitter 913, and the motion sensor 914.

FIG. 11 is a bottom perspective view of the second exemplary embodiment configured for installation on generic surfaces, displaying the device 900, the fastener surface 910, and the battery 911.

FIG. 12 is a front-elevated view of the second exemplary embodiment configured for installation on a urinal, displaying the device 1200, the emitters 1210, and the motion sensor 1211.

FIG. 13 is a rear-lowered view of the second exemplary embodiment configured for installation on a urinal, displaying the device 1200, the fastener surface 1212, and the batteries 1213.

FIG. 14 is a side-perspective view of the second exemplary embodiment configured for installation on a urinal, displaying the device 1200, the emitters 1210, and the motion sensor 1211.

### DETAILED DESCRIPTION OF THE INVENTION

Referring now to the invention in more detail, the invention is directed to a sanitary device 10, which is intended for sanitizing the floor area in front of and around a toilet, urinal, other restroom fixture, or other fixed area where active, automated sanitization in immediate response to use is desirable. The device 10 is equipped with at least one sanitizing emitter 11. In the first exemplary embodiment, a mist of quick-drying sanitizing solution is sprayed onto the floor via the emitter 11 in the area immediately in front of and surrounding the fixture. In the second exemplary embodiment, the device 10 contains a refillable or replaceable bladder, bag, or similar reservoir 15 of sanitizing solution. In a second exemplary embodiment, the emitter 10 is an ultraviolet germicidal irradiation (UVGI) lamp, and the mechanical components related to storage and distribution of a solution are omitted. Generically, either of the two types of emitters may be referred to as a “germicidal emitter”.

The device 10 is preferably flexible, and may be contoured by the user for secure mounting on the lower front surface of a restroom fixture, wall, or other surface. One or more motion sensors 11, located on the front surface of the body 10A of the device 10 detect moving objects, such as the legs and feet of the user, in the floor area immediately in front of the fixture. The device is configured such that, whenever a moving object is detected and then the motion stops for a suitable period, such as five seconds, it will indicate that a user has used the fixture and then left. In the first exemplary embodiment, the device 10 responds by spraying a fine mist of sanitary solution from a refillable or replaceable bladder, bag, or similar reservoir 15 onto the floor area in a semicircular arc, preferably from 120° to 240°, and at a radius of one to five from the device 10. The device 10 is similarly configured for automatic, cyclical operation which may be established by the user for any suitable frequency, such as once every two hours from 8:00 AM to 8:00 PM. Suitable software and electronics for implementing the above-described behavior are well-known in the prior art.

The motion sensor 11 preferably is an infrared motion detector, which detects movement, distance of the moving object, and optionally body heat to detect when the user has departed the area. Such devices are well-known in the prior art. Formally, the motion sensor 11 is configured so as to respond to the presence and then absence of motion consistent with the recent use of the immediate surroundings, for example use of a toilet, urinal, or sink by a human user, followed by the immediate absence of any person.

The emitter 12, mounted on the bottom surface of the body 10A of the device 10 in the first exemplary embodiment, and the motion sensor 11 preferably have a maximum 240° operational arc. While the device 10 is intended for use to eliminate bacteria present in urine, it is to be understood that this is not intended to be a limitation. The device 10 may be used to eliminate, control, and contain any similar surface contaminant, for which quick, reliable, and sanitary elimination and containment are of paramount importance. The entire device 10 is preferably water resistant, preventing any entry of incidental water, urine, or other foreign liquids from the exterior of the device 10 which may contaminate the interior of the device 10, without the device 10 being required to be able to be submerged.

In the first exemplary embodiment, the emitter 12 is a spray nozzle, and is mounted on a rotating mechanism 12A and an adjustable ball joint 12B, and sprays the sanitary solution in a fine mist.

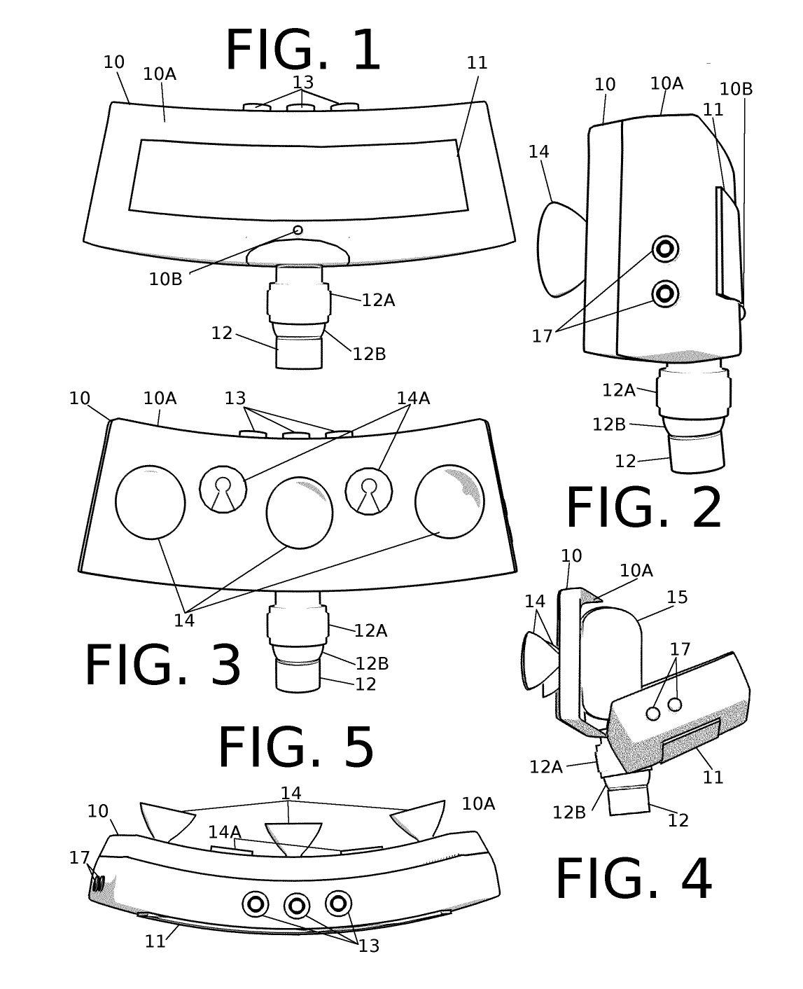

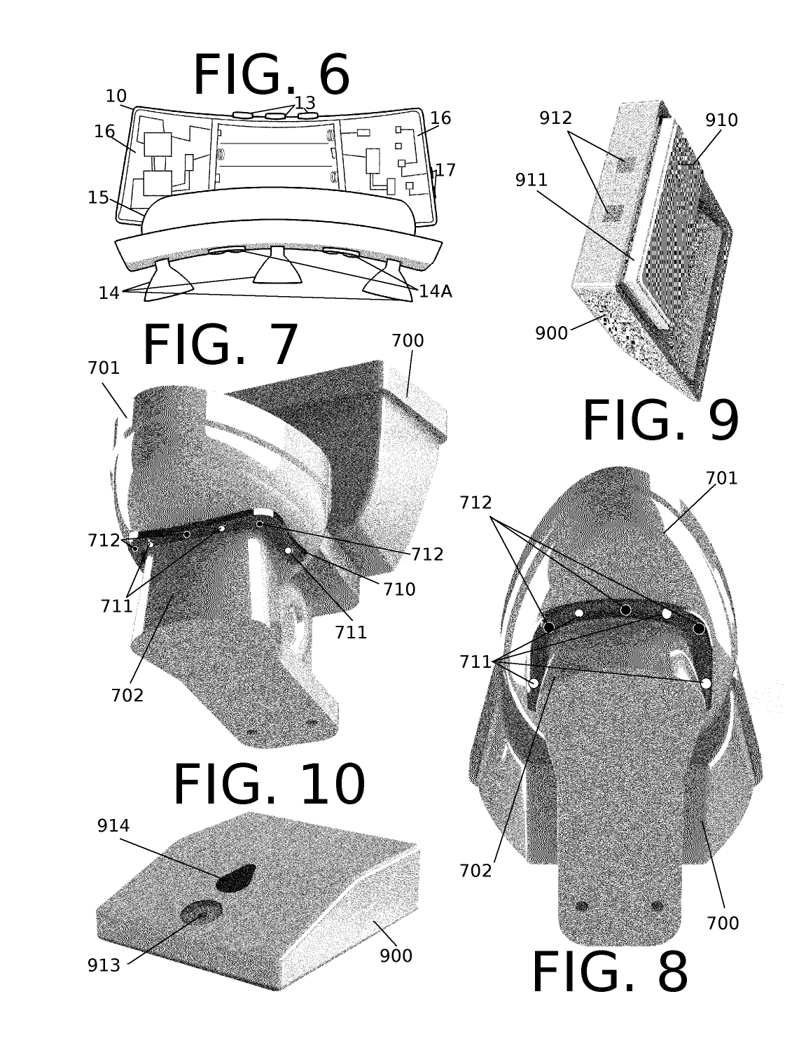

The device 10 preferably may be affixed to a surface of installation using a plurality of fasteners 14 to affix the body 10A to the lower front surface of a restroom fixture, or to the wall immediately below a wall-mounted fixture; generically, toilets (for example, the standard toilet 700), urinals, sinks, stall dividers, walls, and other surfaces may be understood as “mounting surfaces”. In the first exemplary embodiment as shown in FIGS. 1-6, the fasteners 14 are suction cups, as shown. Additionally, secondary fasteners 14A may be provided in addition the primary fasteners. In the case of the first exemplary embodiment, secondary fasters 14A consisting of mechanical mounts to be rested upon or secured to screws, nails, bolts, or the like. Alternate fasteners 14 are also contemplated, for example a repositionable adhesive, static cling structures, etc. In the several forms of the second exemplary embodiment herein presented, a fastening surface 910 or 1212 is shown by example and not by way of limitation. It is generally preferred and understood that the fasteners 14 be configured to allow for tool-less detachment and reattachment from the mounting surface to facilitate cleaning and maintenance of the device and recharging of an onboard battery 911 or 1213. Where the fasteners 14 are used in conjunction with secondary fasteners 14A, and where the fasteners 14 are not suitable for adhering to a particular wall surface substrate, the fasteners 14 are preferably removable such that the secondary fasteners 14A are exposed. Where batteries are used, whether replaceable or fixed rechargeable, the battery capacity must be balanced against the overall size of the device. The device preferably has a sufficient battery capacity for an unattended operational life of at least one week between regular maintenance events, which including charging the battery.

The device 10 is controlled by one or more flexibly printed circuit boards 16, which provide the mounting surfaces for the electronic components, including a microprocessor, a memory device, and a power source, for example a rechargeable Lithium-Ion battery 911 or 1213. Where a rechargeable battery is used, the battery may be energized via a pair of charging terminals 912. The circuit boards 16 may feature accessibility by the user via one or more data ports. Preferably, the data ports are of one or more of a variety of designs which are well known to one practiced in the art. An alternative power source of the first exemplary embodiment may be four replaceable AA batteries, although embodiments featuring more or fewer replaceable batteries, batteries of a different size, rechargeable batteries as described above, or the use of AC wall current as their power sources are also contemplated. If powered by one or more batteries, the device 10 preferably also features a low battery indicator light 10B, preferably located immediately below the motion sensor 11, and consisting of a visible light emitting diode.

In the first exemplary embodiment, the front surface is affixed to the body 10A via two or more clips along the lower edge. The front surface contains the motion sensor 11, the printed circuit boards 16, and the side control buttons 17. The device 10 is capable of two modes. In the inspection mode, the unit is not functional and the front surface has been lowered, opening the body 10A. Inspection mode is used for inspection, maintenance, and repair purposes. By closing and then latching the front surface, the user transitions the device 10 to operational mode.

The device 10 preferably also features top control buttons 13, which are located on the top surface of the body 10A, and side control buttons 17, which are located on the side surface of the body 10A. The top control buttons 13 preferably include controls for the range of the motion sensor 11, preferably from one foot to six feet in six-inch increments; the radius of the motion sensor 11, preferably from 90° to 240°; and a test button. The side control buttons 17 preferably include a manual override and an on/off switch, for periods when the building is vacant, or other periods when the restroom is not in use.

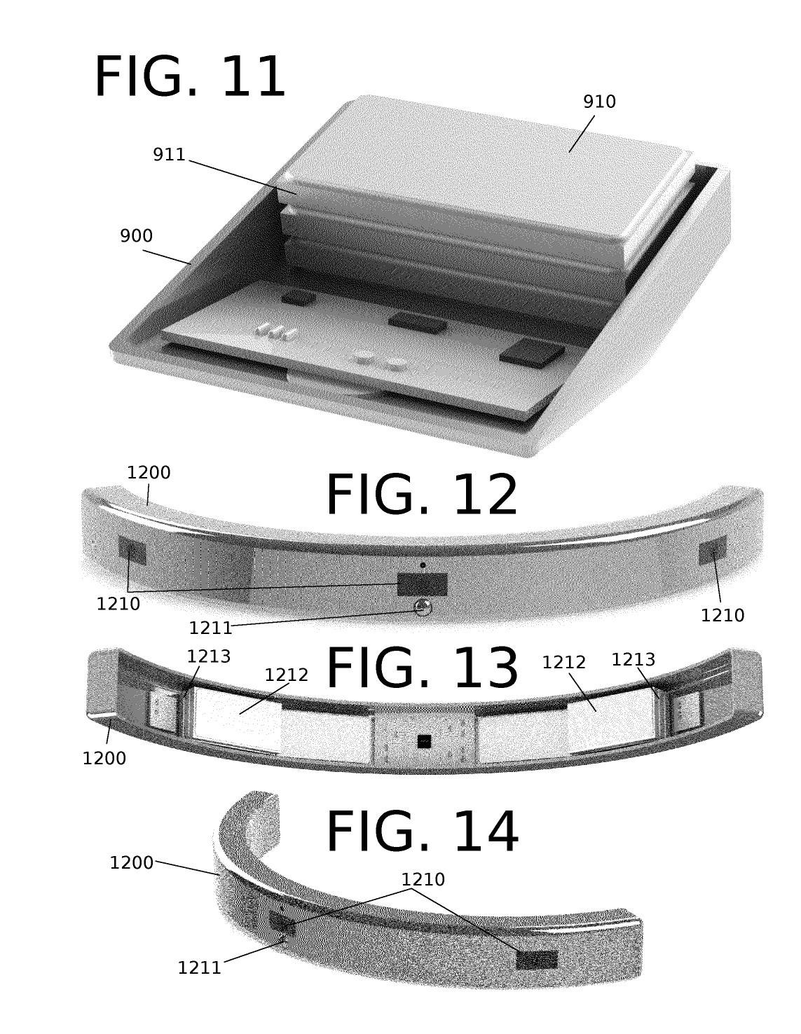

The second exemplary embodiment of FIGS. 7-14 has as its emitter an ultraviolet germicidal irradiation (UVGI) lamp or LED 711, 913, or 1210, which replaces the spray of sanitizing solution (generically, an “ultraviolet emitter”). In the second exemplary embodiment, the spray nozzle-type emitter 12, the rotation mechanism 12A, the ball joint 12B, and the reservoir 15 are replaced by the ultraviolet emitter, which is installed in the same position as the spray nozzle 12, or as shown in the alternative forms. In the second exemplary embodiment, the ultraviolet emitter may be formally understood to respond to the motion sensor by irradiating the surroundings of the device, in a generally downward direction, with UV light for a predetermined period and at a predetermined intensity that is germicidally effective as to the surroundings. In all other respects, the first and the second exemplary embodiments may be operated identically. Alternatively, the UVGI lamp and spray system may be combined into a hybrid embodiment utilizing both sanitization methods. Generically, the action of UV light and the action of the spray nozzle-type emitter 12 are referred to as “causing the emitter to sanitize the surroundings”, irrespective of the mechanism or combination of mechanisms used.

The UVGI lamp or LED, or other ultraviolet emitter produces Ultraviolet C (“UVC”, or, in the claims, “ultraviolet c”), with a wavelength range, in nanometers, of 280-100 nm, and energy, per photon, of 4.43-12.4 eV. The ultraviolet emitter preferably contains no fluorescent phosphor and its exterior is preferably manufactured from fused quartz. The lamp tube, if provided, is similar to an ordinary fluorescent lamp, preferably with a wavelength of 253.7 nm, and the LED is preferably similar to a visible LED in one of a variety of colors. In an alternate embodiment, the ultraviolet emitter may be in addition to, or supplement, the currently specified decontamination spray apparatus of the first exemplary embodiment.

The light produced by the ultraviolet emitter is preferably filtered to remove as much visible light as possible, leaving only the UVC light. Preferably, to reduce or eliminate ozone emission, the ultraviolet emitter is treated to absorb the 185 nm mercury emission line, which is the longest wavelength of mercury light which will ionize oxygen. Since the viewing of UV light is harmful, the light produced by the ultraviolet emitter is well-shielded against both direct viewing and then viewing of reflections by any user, entirely dispersed downward to the floor area, and configured to only emit in the reasonable likelihood of the absence of a human user, as interpreted through the motion sensors 712, 913, and 1211.

As pictured, the second exemplary embodiment may be presented in a variety of structural forms. FIGS. 7-8 show a u-shaped device 710 affixed to a standard toilet 700 at the interface between the toilet bowl 701 and the toilet base 702. The u-shaped 710 device surrounds the toilet bowl 701 on three sides, and covers the surrounding floor area with a plurality of emitters 711 and motion sensors 712.

In the second exemplary embodiment, a generic form of the device 900, shown in FIGS. 9-11 is oblong in footprint and combines a single emitter 913 with a single motion sensor 914. The generic form provides a rechargeable Lithium-Ion battery 911, recharging ports 912, and is mounted to any desired surface via the fastening surface 910, which may take any of the above-described fastener forms.

An arc-shaped form of the second exemplary embodiment, as shown in FIGS. 12-14, provides a device 1200, emitters 1210, a motion sensor 1211, fastener surfaces 1212, and rechargeable Lithium-Ion batteries 1213.

To use the first and second exemplary embodiments, the user opens the body 10A by unlatching and lowering the front surface of the body 10A, transitioning the device 10A to inspection mode. The front surface is affixed to the body 10A via one or more hinges along the lower edge. The front surface contains the motion sensor 11, the circuit boards 16, and the side buttons 17. Operational parameters and other data may be input into, or copied from, the side buttons 17 by an appropriate external user device, such as a keyboard, flash drive, smartphone, or tablet computer. The user may refill the reservoir 15 of the first exemplary embodiment, and replace the battery or batteries of both exemplary embodiments. The user may then close the front surface of the body 10A, transitioning the device 10 to operational mode. The user may further control the device 10 through the use of the control buttons 13.

The body 10A and the circuit boards 16 are preferably manufactured from semi-rigid, durable materials, such as plastic, rubber or silicone, and the devices of the second exemplary embodiment 700, 900, and 1200 may be of similar materials. The cover of the motion sensors 11, 712, 914, and 12 is preferably manufactured from a semi-rigid, durable, transparent, or translucent material which is easily cleaned, such as plastic, and the motion sensors themselves are preferably of common, well-known, commercially available variety. The UV light source emitters 711, 913, and 1210 are preferably commercially available and well-known ultraviolet LEDs or other types of ultraviolet light sources. The reservoir 15 of the first exemplary embodiment is preferably manufactured from a flexible, durable material, such as plastic. The fasteners 14, where suction cups are provided, are preferably manufactured from a flexible, durable material such as rubber or silicone. The spray nozzle 12, the top control buttons 13, and the side control buttons 17 of the first exemplary embodiment are preferably manufactured from rigid, durable materials such as plastic, fiberglass, steel, and aluminum. Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

We claim:

1. A floor sanitizing device comprising:
(a) a device;
(b) said device being equipped with at least one germicidal emitter;
(c) said device being equipped with at least one motion sensor;
(d) a plurality of fasteners being affixed to the exterior of said device;
(e) said plurality of fasteners affixing said device to a mounting surface.
(f) said device being configured so as to respond, as detected by said at least one motion sensor, to the presence and then absence of motion consistent with the recent use of the immediate surroundings of said device followed by the immediate absence of any person by causing said at least one germicidal emitter to sanitize the surroundings of said device.

2. The floor sanitizing device of claim 1 wherein:
(a) said at least one germicidal emitter is an ultraviolet emitter;
(b) said ultraviolet emitter being configured so as to emit light in the ultraviolet c band;
(c) said ultraviolet emitter being configured such that, when said ultraviolet emitter is caused to sanitize the surroundings of said device, said ultraviolet emitter emits ultraviolet light for a predetermined period and at a predetermined intensity that is germicidally effective as to the surroundings of said device.

3. The floor sanitizing device of claim 2 wherein said mounting surface is a standard toilet; said standard toilet having a bowl affixed over a base; said device is u-shaped; and said device is affixed about said bowl.

4. The floor sanitizing device of claim 2 wherein said device has an oblong footprint, and wherein said device is suitable as to said mounting surface, generically.

5. The floor sanitizing device of claim 2 wherein said device is arc-shaped and wherein said device is suitable as to being affixed to urinals.

6. The floor sanitizing device of claim 1 wherein said device further comprises a battery; said battery being configured so as to provide power to said at least one motion sensor and said at least one germicidal emitter; said battery being permanently installed and rechargeable; said battery providing said device with an unattended operational life of at least one week.

7. The floor sanitizing device of claim 2 wherein said device further comprises a battery; said battery being configured so as to provide power to said at least one motion sensor and said at least one germicidal emitter; said battery being permanently installed and rechargeable; said battery providing said device with an unattended operational life of at least one week.

8. The floor sanitizing device of claim 3 wherein said device further comprises a battery; said battery being configured so as to provide power to said at least one motion sensor and said at least one germicidal emitter; said battery being permanently installed and rechargeable; said battery providing said device with an unattended operational life of at least one week.

9. The floor sanitizing device of claim 4 wherein said device further comprises a battery; said battery being configured so as to provide power to said at least one motion sensor and said at least one germicidal emitter; said battery being permanently installed and rechargeable; said battery providing said device with an unattended operational life of at least one week.

10. The floor sanitizing device of claim 5 wherein said device further comprises a battery; said battery being configured so as to provide power to said at least one motion sensor and said at least one germicidal emitter; said battery being permanently installed and rechargeable; said battery providing said device with an unattended operational life of at least one week.

11. The floor sanitizing device of claim 1 wherein said plurality of fasteners is configured so as to permit tool-less detachment and re-attachment of said device from and to said mounting surface.

12. The floor sanitizing device of claim 2 wherein said plurality of fasteners is configured so as to permit tool-less detachment and re-attachment of said device from and to said mounting surface.

13. The floor sanitizing device of claim 3 wherein said plurality of fasteners is configured so as to permit tool-less detachment and re-attachment of said device from and to said mounting surface.

14. The floor sanitizing device of claim 4 wherein said plurality of fasteners is configured so as to permit tool-less detachment and re-attachment of said device from and to said mounting surface.

15. The floor sanitizing device of claim 5 wherein said plurality of fasteners is configured so as to permit tool-less detachment and re-attachment of said device from and to said mounting surface.

16. The floor sanitizing device of claim 6 wherein said plurality of fasteners is configured so as to permit tool-less detachment and re-attachment of said device from and to said mounting surface.

17. The floor sanitizing device of claim 7 wherein said plurality of fasteners is configured so as to permit tool-less detachment and re-attachment of said device from and to said mounting surface.

18. The floor sanitizing device of claim 8 wherein said plurality of fasteners is configured so as to permit tool-less detachment and re-attachment of said device from and to said mounting surface.

19. The floor sanitizing device of claim 20 wherein said plurality of fasteners is configured so as to permit tool-less detachment and re-attachment of said device from and to said mounting surface.

20. The floor sanitizing device of claim 20 wherein said plurality of fasteners is configured so as to permit tool-less detachment and re-attachment of said device from and to said mounting surface.
